(19) United States
(12) Patent Application Publication
(10) Pub. No.: US 2009/0132055 A1
(43) Pub. Date: May 21, 2009

(54) ARTHROPLASTY DEVICE

(76) Inventor: Thomas D. Ferro, Arroyo Grande, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/268,167

(22) Filed: Nov. 10, 2008

Related U.S. Application Data
(60) Provisional application No. 60/986,340, filed on Nov. 8, 2007.

(51) Int. Cl. A61F 2/38

(52) U.S. Cl. 623/20.21; 623/20.35; 623/20.32; 623/20.31

(57) ABSTRACT

Disclosed is a device and method of tricompartmental arthroplasty of the knee. The device permits arthroplasty of the medial and lateral and patellofemoral compartments of the knee while leaving the anterior and posterior cruciate ligaments intact. The device provides a femoral implant component that includes a trochlear surface and tibial prostheses component which can be secured to the tibia.

### REFERENCE TO PRIORITY DOCUMENT

This application claims priority of co-pending U.S. Provisional Patent Application Ser. No. 60/986,340 entitled “Arthroplasty Device”, filed Nov. 8, 2007. Priority of the aforementioned filing date is hereby claimed, and the disclosure of the Provisional Patent Application is hereby incorporated by reference in its entirety.

### BACKGROUND

This disclosure relates to a device and method for tricompartmental arthroplasty for resurfacing of both the medial joint and the lateral in addition to the patellofemoral joint of the knee in order to treat the pain associated with arthritis. In an embodiment, the present device and method resurfaces both the medial and lateral joints, or just the medial or lateral joint and the corresponding half of the trochlea of the patellofemoral joint, depending on which compartment is diseased. A one piece embodiment resurfaces both the lateral (outside) and medial (inside) compartment of the knee at the same time, as well as the trochlea of the patellofemoral joint.

Total knee joint replacement (arthroplasty) is a common and very successful surgery for people with degenerative arthritis (osteoarthritis) of the knee. Over 300,000 patients a year in the United States undergo total knee replacement surgery. Total knee replacement puts an artificial surface on all parts of the knee joint that contact each other as the knee bends. The damaged cartilage is removed, a measured amount of bone is removed, the knee implant is attached to the distal end of the femur and proximal end of the tibia, and the patella is resurfaced if necessary. The implant typically is made of metal and plastic and provides an artificial articulating surface which causes no pain to the patient.

Unfortunately total knee replacement results in significant bone resection. Anterior and posterior chamfer cuts are made from the distal femur followed by a posterior femoral cut which results in an octagonal shape of the end of the femur, with the removal of significant bone of varying depth of cut. Additionally, knee arthroplasy requires sacrifice of the anterior cruciate ligament and sometimes the posterior cruciate ligament as well.

More recently, unicompartmental knee arthroplasty has been utilized where there is arthritic damage to only a single compartment of the knee and no damage to the other compartments. The inside (medial) component (medial tibial plateau and the medial femoral condyle) is most commonly involved and replaced using unicompartmental arthroplasty. However, occasionally, the outside (lateral) compartment (the lateral tibial plateau and the lateral femoral condyle) is sometimes involved and must be replaced. Also the knee cap, i.e., the patellofemoral compartment (the patella and femoral trochlear notch) may also develop osteoarthritis. Heretofore, if more than a single compartment of the knee had arthritic disease, total knee replacement was the only available treatment.

### SUMMARY

Disclosed is a device and method of tricompartmental arthroplasty of the knee. The device permits arthroplasty of the medial and lateral and patellofemoral compartments of the knee while leaving the anterior and posterior cruciate ligaments intact. The device provides a femoral implant component that includes a trochlear surface and tibial prostheses component which can be secured to the tibia. In an embodiment, the femoral component may be a uniform thickness resurfacing geometry which decreases bone resection, and is available as a single resurfacing unit, a two piece modular unit separated down the trochear groove, and as a modular unit with a separate railing underneath that fixates to the bone by cement (in CrCo steel alloy) or through bone attachment (osseointegration via rough titanium surface).

The modular two piece femoral component may include a separate medial and lateral components that can be independently mixed for optimal reproduction of the patients own anatomy and size. All of the femoral components may have enlarged trochlear cutouts, and narrowed profiles to allow preservation of the cruciate ligaments and peripheral bone. The femoral design is of a thinner surface with a deepened groove running centrally, or peripherally (in the case of the modular option). The femoral component also has a convex articulating surface which engages a concave articulating surface of the tibial implant component to approximate the articulation of a healthy knee.

Other features and advantages should be apparent from the following description of various embodiments, which illustrate, by way of example, the principles of the invention.

### BRIEF DESCRIPTION OF THE FIGURES

FIG. 1 shows a perspective view of a single-piece or monoblock implant system.

FIG. 2 shows a front view of the monoblock implant system.

FIG. 3 shows a right view of the monoblock implant system.

FIG. 4 shows a top view of the monoblock implant system.

FIG. 5 shows a left view of the monoblock implant system.

FIG. 6 shows a perspective view of a two-piece implant system.

FIG. 7 shows a front view of the two-piece implant system.

FIG. 8 shows a right view of the two-piece implant system.

FIG. 9 shows a back view of the two-piece implant system.

FIG. 10 shows a top view of the two-piece implant system.

FIG. 11 shows a left view of the two-piece implant system.

### DETAILED DESCRIPTION

Before the present subject matter is further described, it is to be understood that this subject matter described herein is not limited to particular embodiments described, as such may of course vary. It is also to be understood that the terminology used here in is for the purpose of describing particular embodiments only, and is not intended to be limiting. Unless defined otherwise, all technical terms used herein have the same meaning as commonly understood by one skilled in the art to which this subject matter belongs.

As will be apparent to those of skill in the art upon reading this disclosure, each of the individual embodiments described and illustrated herein has discrete components and features which may be readily separated from or combined with the features of any of the other several embodiments without departing from the scope of the subject matter described herein. Any recited method can be carried out in the order of events recited or in any other order which is logically possible.

The present disclosure provides a device and method for tricompartmental arthroplasty which permits the resurfacing of the medial and lateral and patellofemoral joints of the knee or just the medial joint and lateral half of the patellofemoral joint, or just the lateral and lateral half of the patellofemoral joints (without the necessity for resurfacing of the opposite compartment of the knee), and without the sacrifice of the anterior or posterior cruciate ligaments. Additionally, a two piece femoral component prosthesis allows for independent sizing of the medial and lateral half of the joint, as different sizes are compatible because of the standardized dimensions of the patellofemoral groove (trochlea) formed by the adjacent placement of medial and lateral femoral prosthesis components.

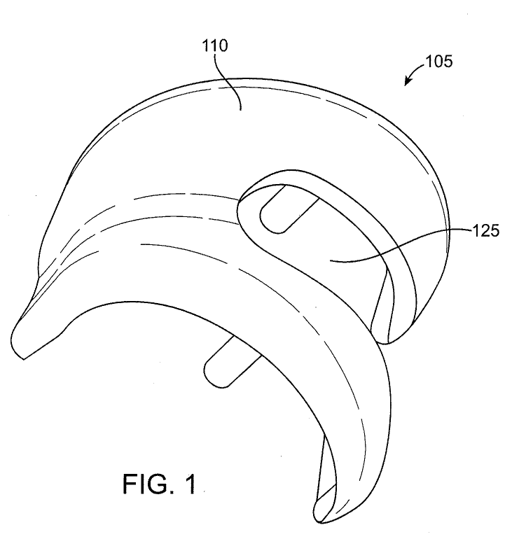

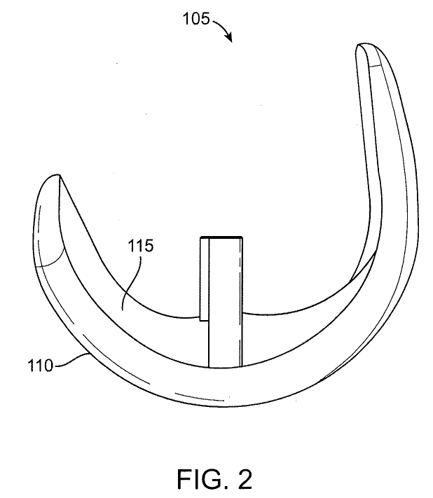

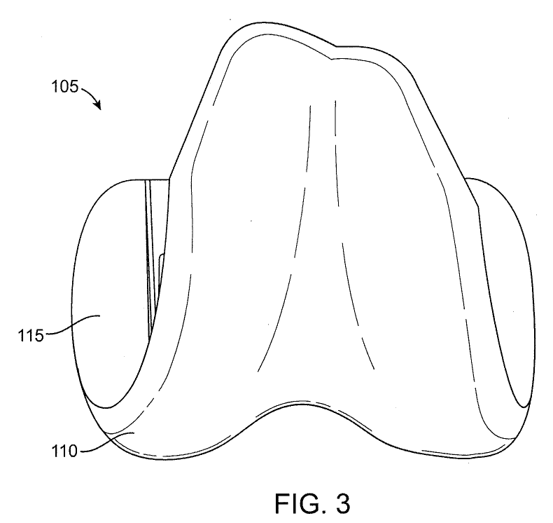

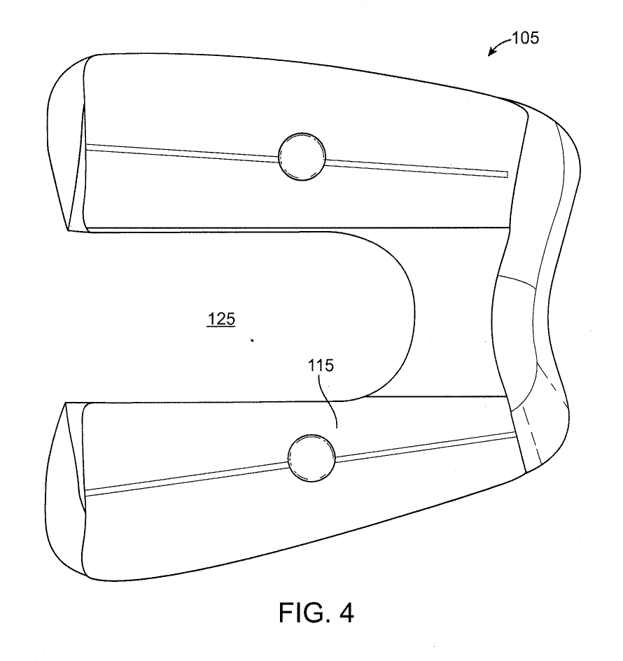

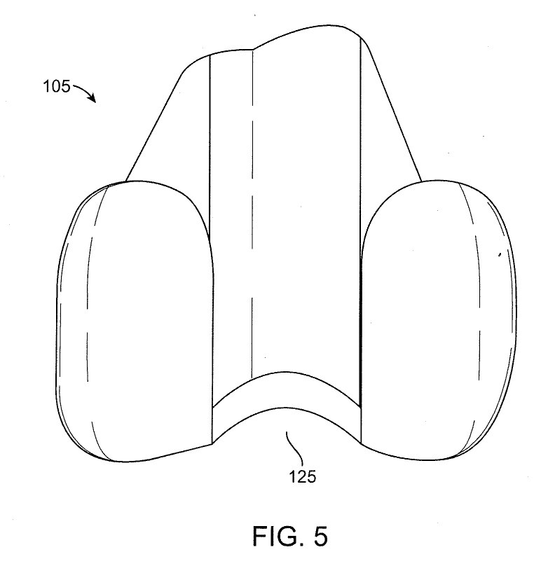

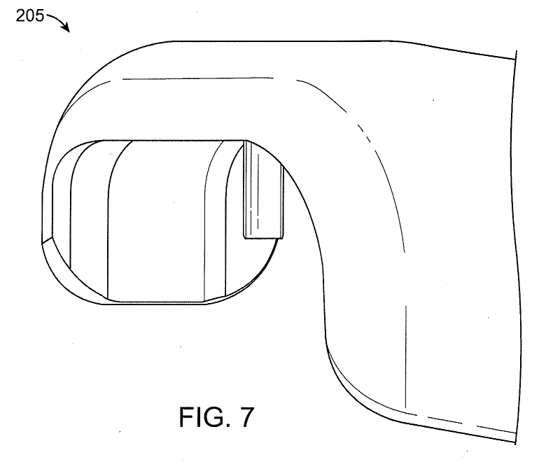

FIGS. 1-7 show various views of a femoral resurfacing implant 105 of the invention suitable for implantation at the knee joint. The implant 105 has an upper surface 110, a lower surface 115 and a peripheral edge. The implant 105 includes a c-shaped cut-out or opening 125 positioned down the centerline of the implant. The implant 105 can be formed of a single, monolithic piece of material, or the implant 105 can be formed of an outer shell that mates to an inner shell, such that the outer and inner shell collectively form the implant.

In an embodiment, a femoral portion of the implant 105 (the cap that resurfaces the end of the femur) comprises a thin unique geometry with a constant inner side curvature. A cutting machine, such as a robotic cutting machine, can be used to easily cut complex curvatures into the bone that allow the precise removal of only that bone that is replaced by the implant 105. This allows resurfacing of the end of the femur bone with significantly less bone removal that any other knee replacement, approximately half the bone removal. This also avoids the stress risers that all current total knee femoral components experience at the junctions of the flat inner surfaces of the components.

The implant 105 is less wide than conventional implants, with a larger portion removed to allow for less removal of bone from the peripheral edge on the bone end surface, and with more of a cutout to allow for preservation of the ACL. The implant 105 also has a unique inner surface geometry that provides for a central fin that runs the length of the inner curve which fits over the medial and lateral femoral condyles, respectively (similar to unicondylar prostheses, but longer) This improves fixation of the prosthesis, especially for a porous coating is used for fixation via bone ingrowth or ongrowth (osseointegration) rather than cement fixation. It also strengthens the prosthesis to resist bending forces experienced in the “C” shaped portions that resurface the end of each condyle.

The implant 105 has a unique posterior condylar geometry in which the implant curves upward moving distal to proximal (up the back of the knee towards the hip) causing it to require a rolling action (from posterior distal to anterior proximal) for implantation because of an inward cant of the planes or the inner surface of the anterior and posterior surfaces of the component. The anterior and posterior surfaces are not parallel, but rather planes that will eventually intersect if carried far enough in a proximal direction (up the thighbone).

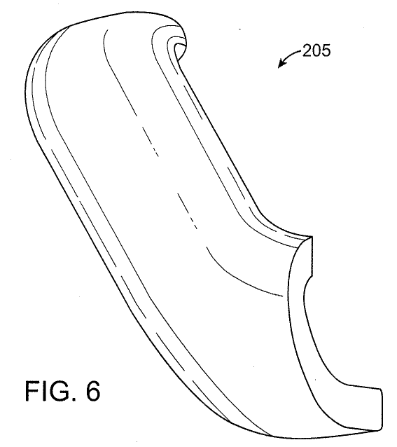

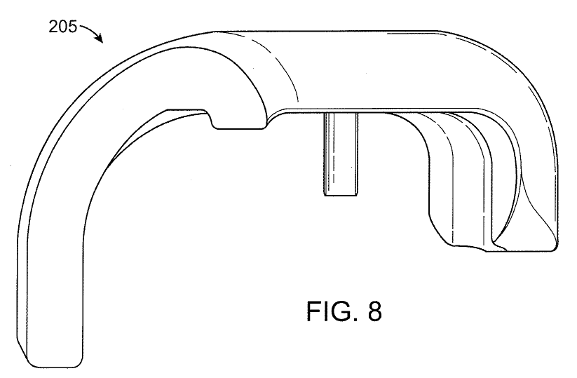

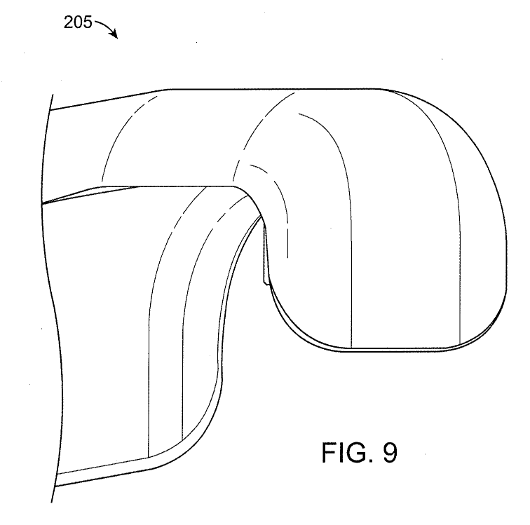

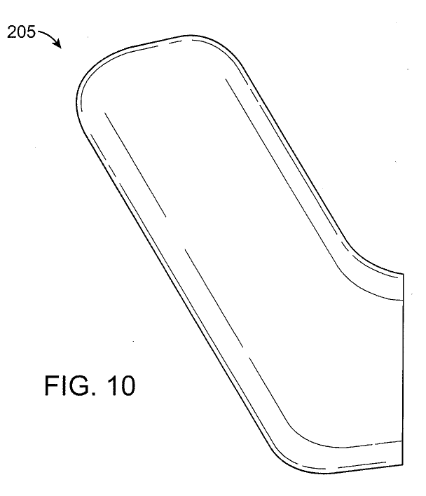

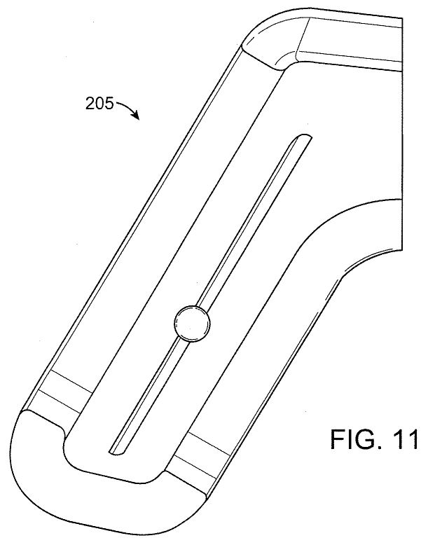

In another embodiment, the implant is a modular two-piece system. FIGS. 6-11 show various views of one piece 605 of the two-piece system. The other piece would be a mirror of the piece 605. The two piece system is essentially like the one piece implant 105 (shown in FIGS. 1-5) cut down the middle (right through the trochlear groove, with the edges beveled). The geometry is otherwise essentially identical to the one-piece implant 105. The two piece system allows one to size the medial half and lateral half of the end of the femur independently, and even allows one to resurface only one half or the other in cases when only one half is diseased. The precise positioning by of the component in the bone by the robot allows for resurfacing of only one half of the patellar groove, allowing for a smooth transition from the prosthesis to the unresurfaced half of the patellar groove (or trochlea).

When implanted in the body, the two halves of the two-piece implant do not in any way attach or connect; they are precisely placed with a gap, such as a 1-2 mm gap between the two pieces. This prevent wear, fretting of materials, or failure of the device at the trochlea, as the “C” shaped medial and lateral components are inherently structurally strong designs.

Although there are conventional 3 piece designs for femoral components for total knees, these are difficult to implant and the pieces do not have the inherent stability of a monoblock component when placed on the bone. The 2-piece system retains the inherent stability of a standard total knee femur component, that of a “C” shape fitting over the end of a prepared surface, developing compressive forces from front (anterior) to back (posterior).

The prosthesis also has a constant curvature of radius on the outer surface on the medial condyle (the middle half of the end of the femur), as well as propriety trochlea groove dimensions.

The same monoblock and 2 piece femoral resurfacing implants can be made in a unique manner with a modular undersurface component that attaches directly to the bone, via bone ingrowth or ongrowth (made of titanium alloy, TiAlNo), or via cement (made of Cobalt-Chromium Steel alloy, CrCo). The outer, exterior surface that attaches to the undersurface via a press fit of trapezoidal surfaces, similar to the morris taper utilized to fit hip prosthesis balls to stem neck components. The modular undersurface component is fit into the bone via precise machining of the bone, such as by a robotic milling device, and can then accept different size monoblock or two piece modular exterior component surfaces, which have the unique geometries as specified above. The 2 piece femoral implants can also be individually made with these modular fixation designs, to resurface only the medial femoral condyle and medial half of the patellar groove (trochlea), or only the lateral femoral condyle and lateral half of the patellar groove (trochlea).

In an embodiment, a device for tricompartmental arthroplasty of a patient's knee comprises a femoral prosthesis component configured to resurface the patellofemoral and both of the other compartments of the knee, with a constant thickness of material which requires less removal of bone than in standard knee arthroplasty, and with reduce design width to allow room for retention of the Anterior Cruciate Ligament without impingement of the anterior margin of the prosthesis component of the femur. Additionally the prosthesis can have various embodiments, including a single piece or two modular femoral prosthesis component pieces. The two pieces are configured corresponding to the geometry of the medial or lateral half of the distal end femur and configured to resurface either the medial compartment or the lateral compartment, and the medial half of the patellofemoral joint or the lateral half of the patellofemoral joint, depending on which compartment is diseased.

The femoral prosthesis component has a first internal surface configured in a continuous radius to be secured to a surgically prepared distal end of the one other compartment of the patient's femur so that the anterior and posterior cruciate ligaments remain intact. The femoral implant component also has a second exterior convex curved surface positioned and configured to replicate a femoral condyle for that compartment and a concave trochlea surface positioned and configured to articulate with the patella. The device also comprises tibial implant components configured for the other compartments of the knee having a first interior surface configured to be secured to a surgically prepared proximal end of the one other compartment of the patent's tibia and a second concave curved or flat exterior surface configured to receive the second convex curved surface of the femoral implant component to permit pivotal and gliding articulation between said femoral implant component and the tibial implant component, approximating the articulation of a healthy knee joint.

A method of performing tricompartmental arthroplasty of the knee with reduced removal of bone, with modular options for the medial and lateral femoral components, and with modular options for the fixation of the outer surface to the bone comprises the steps of making a 3 to 4 inch longitudinal incision on the anterior aspect of the knee from the medial aspect of the patellar tendon 1 centimeter below the joint line extending approximately 3 to 4 inches in a vertical fashion. The incision is opened along the length of the incision with a medial parapatellar arthrotomy extending in a minimal fashion into the quadriceps femoris muscle medially. The remnants of the medial meniscus are excised.

A portion of the fat pad is excised with care being taken to preserve the integrity of the anterior and posterior cruciate ligaments, the distal ends of the patient's femur is surgically prepared to receive a femoral implant component utilizing specialized and or robotically controlled precision cutting devices to remove only that bone which will be precisely replaced by the implant component or components in the case of the modular components. The femoral implant component has a concave trochlea surface and a concave first surface configured to be secured to the surgically prepared distal end of the femur. The femoral component also has a second convex curved surface configured to replicate the condyle and articulate with the second concave curved surface of the tibial implant component to permit pivotal articulation between the femoral implant component and the tibial implant component in a manner approximating the articulation of a healthy knee joint.

The tibial compartments are surgically prepared to receive a tibial implant component in a manner so as to preserve the integrity of the anterior and posterior cruciate ligaments. The tibial implant component has a first surface configured to be secured to the surgically prepared one other compartment of the proximal end of the tibia and a second concave curved surface. The femoral component is next secured to the femur and the tibial component is secured to the tibia. Next the patella is rotated approximately 90 degrees. The patella is positioned over the concave trochlea surface of the femoral implant component and the incision is closed.

If the patella is also diseased, the method also comprises the additional steps of surgically preparing a posterior surface of the patent's patella to receive a patella implant component having a first surface configured to be secured to the surgically prepared posterior surface of the patella, and a convex curved second surface configured to engage the concave trochlea surface of the femoral implant component, and securing the patella implant component to the patella. A partial lateral facetectomy is typically performed to limit the patellar contact with the transition zone of the distal femur and the femoral component. The patellar implant should be medialized to assist with patella tracking. Release of the medial collateral ligament can be employed as necessary to allow for ligamentous balancing of the arthroplasty

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and endoscope of the appended claims should not be limited to the description of the embodiments contained herein.

What is claimed:

1. A device for tricompartmental arthroplasty of the knee comprising:
monolithically formed medial and lateral femoral implant components having a first internal concave surface configured to be secured to surgically prepared medial and lateral compartments of a distal end of a patient's femur and a second exterior convex curved surface positioned and configured to replicate a normal medial and lateral femoral condyle;
said medial and lateral femoral implant components also having a concave trochlea surface formed at the approximate junction of the two components, so positioned and configured to articulate with the patella; and
unicondyle medial and lateral tibial implant components having a first interior surface configured to be secured to a surgically prepared media and lateral compartments of a proximal end of the patient's tibia and a second flat or concave curved exterior surface configured to receive the second convex curved surface of said medial and lateral femoral implant components to permit gliding and pivotal articulation between said medial and lateral femoral implant components and said medial and lateral tibial implant components, said pivotal and gliding articulations approximating the articulations of a healthy knee joint.

2. A device for tricompartmental arthroplasty of the knee as claimed in claim 1 wherein said medial and lateral femoral implant components are made of metallic material.

3. A device for tricompartmental arthroplasty of the knee as claimed in claim 1 wherein said medial and lateral femoral implant components are made of a bio-compatible plastic.

4. A device for tricompartmental arthroplasty of the knee as claimed in claim 1 wherein said medial and lateral tibial implant components are made of a bio-compatible plastic.

5. A device for tricompartmental arthroplasty of the knee as claimed in claim 1 wherein said medial and lateral tibial implant components are made of metallic material.

6. A device for tricompartmental arthroplasty of the knee as claimed in claim 1 wherein said medial and lateral tibial implant components are made of both a bio-compatible plastic and metallic material.

7. A device for tricompartmental arthroplasty of the knee as claimed in claim 1 wherein said medial and lateral femoral implant components and said medial and lateral tibial implant components are configured and dimensioned to permit the distal end of the femur and the proximal end of the tibia to be surgically prepared in manner so as to preserve the integrity of the anterior and posterior cruciate ligaments.

8. A device for tricompartmental arthroplasty of the knee as claimed in claim 1 wherein the device is configured and dimensioned to permit the distal end of the femur and the proximal end of the tibia to be surgically prepared while the patella is rotated at approximately 90 degrees.

9. A device for tricompartmental arthroplasty of the knee as claimed in claim 1 further comprising a patella implant component having a first surface configured to be secured to a surgically prepared posterior surface of the patella, and a convex curved second surface configured to articulate with the concave trochlea surface formed by at adjacent placement of the medial and lateral femoral implant components, or in the case of the single femoral component, with the anterior surface of the modular or monolithic one piece surface replacing femoral implant component.

10. A device for tricompartmental arthroplasty of the knee as claimed in claim 9 wherein said patella implant component is configured to be secured to the patella by cement.

11. A device for tricompartmental arthroplasty of the knee as claimed in claim 9 wherein said patella implant component is configured to be secured to the patella by bone ingrowth into an ingrowth surface on the first internal surface of the patella implant component.

12. A device for tricompartmental arthroplasty of the knee as claimed in claim 1 wherein said femoral implant component is configured to be secured to the femur by a cement.

13. A device for tricompartmental arthroplasty of the knee as claimed in claim 1 wherein said femoral implant component is configured to be secured to the femur by bone ingrowth into an ingrowth surface on the first internal surface of the femoral implant component.

14. A device for tricompartmental arthroplasty of the knee as claimed in claim 1 wherein said tibial implant components are configured to be secured to the tibia by a cement.

15. A device for tricompartmental arthroplasty of the knee as claimed in claim 1 wherein said tibial implant components are configured to be secured to the tibia by bone ingrowth into an ingrowth surface on the first interior surface of the tibial implant components.
